(19) United States
(12) Patent Application Publication
(10) Pub. No.: US 2023/0052455 A1
(43) Pub. Date: Feb. 16, 2023

(54) TETHER-FREE ROBOTIC SYSTEM TO PERFORM A REMOTE MICROSURGERY IN THE CENTRAL NERVOUS SYSTEM (CNS)

(71) Applicant: BIONAUT LABS LTD., Herzliya (IL)

(72) Inventors: Florent CROS, Los Angeles, CA (US); Michael SHPIGELMACHER, Los Angeles, CA (US); Kishore Kumar KONDABATNI, Simi Valley, CA (US); Alex KISELYOV, San Diego, CA (US)

(73) Assignee: BIONAUT LABS LTD., Herzliya (IL)

(21) Appl. No.: 17/897,035

(22) Filed: Aug. 26, 2022

Related U.S. Application Data
(63) Continuation of application No. PCT/US2022/040303, filed on Aug. 15, 2022.
(60) Provisional application No. 63/233,652, filed on Aug. 16, 2021.

(51) Int. Cl. A61B 17/00; A61N 2/00

(52) U.S. Cl. CPC A61B 17/00; A61N 2/006; A61B 2017/00876; A61B 2017/00225; A61B 2018/00446

(57) ABSTRACT

The present disclosure relates to systems that comprise a millimeter size tetherless object powered by an external magnetic field, and an interactive hardware-software platform separate from the miniature device that generates, modulates and controls magnetic fields in a defined three-dimensional operational volume to propel, navigate the miniature device to a specific anatomical target to complete a (microsurgical) mission or task, as well as using such systems to perform microsurgery in the central nervous system (CNS).

### CROSS-REFERENCE TO RELATED APPLICATIONS

This application is a Continuation of PCT International Application PCT/US2022/040303, filed Aug. 15, 2022, which claims priority to U.S. Provisional Application No. 63/233,652, filed Aug. 16, 2021, the contents of each of which are hereby incorporated by reference in their entireties.

### FIELD OF THE INVENTION

The subject matter disclosed herein relates to systems that comprise a millimeter size tetherless object powered by an external magnetic field, and an interactive hardware-software platform separate from the miniature device that generates, modulates and controls magnetic fields in a defined three-dimensional operational volume to propel and navigate the miniature device to a specific anatomical target to complete a (microsurgical) mission or task, as well as using such systems to perform microsurgery at the target.

### BACKGROUND OF THE INVENTION

Congenital hydrocephalus is a condition resulting from increased intracranial pressure in the ventricular system due to a derangement between the balance in production and absorption of cerebrospinal fluid (CSF). In children, classical clinical manifestations include bulging fontanelle, persistent downward gaze (“sun-setting” eyes), macrocephaly manifesting as rapidly increasing head circumference, irritability, vomiting, poor feeding, seizures, lethargy, blindness and death. If hydrocephalus is not addressed via neurosurgical intervention, the mortality of the condition is high due to the lethality of this untreated increased intracranial pressure. Dandy-Walker malformation (DWM), Dandy-Walker complex or Dandy-Walker syndrome (DWS) represents a clinical syndrome manifesting as the congenital association of hydrocephalus, posterior fossa cyst, and hypoplasia of the cerebellar vermis. Classic anatomic hallmarks defining DWM are hypoplasia of the cerebellar vermis, anterior-posterior enlargement of the posterior fossa, upward displacement of the torcula and transverse sinuses, and cystic dilatation of the fourth ventricle. Unfortunately, current treatments of DWM that include shunting and endoscopic third ventriculostomy (ETV) produce less than optimal clinical outcomes. These procedures are frequently insufficient, require multiple interventions and carry risks of severe complications including wound healing issues, surgical infections and worsening neurological deficits.

### SUMMARY OF THE INVENTION

Described herein is a platform that performs safe and efficient single or multiple fenestrations of Dandy Walker cyst to balance intracranial pressure in a patient. This system offers an alternative to medical practices that rely on tethered solutions, e.g., catheters, trocars, needles and endoscopes.

Hydrocephalus associated with the typical manifestations of the Dandy-Walker malformation is treated as described herein using microsurgical fenestration of the cyst using a miniature device. Specifically, microsurgical miniature particles that are controlled externally and remotely are designed to perform safe and accurate effective fenestration of the Dandy Walker cyst in posterior fossa in the brain to normalize intracranial pressure. Patients to be treated include pediatric DWM patients 0-6 months old, as well as adolescent and adult patients in whom ETV and/or shunt management of their hydrocephalus is contraindicated due to prior failure from infection, bleeds, and other clinical factors which make shunting or endoscopic interventions unfavorable.

In one aspect, provided herein are miniature devices configured to be directed by an external magnetic field along a path to a target site in the central nervous system (CNS) within a patient, and to perform one or more mechanical actions at the target site under manipulation by an external magnetic field. The miniature devices comprise a body having a head portion and a tail portion defining a longitudinal axis spanning therebetween, the head portion comprising a blade assembly with one or more blades.

In another aspect, provided herein are systems configured to facilitate treatment by microsurgery at a target site in the central nervous system (CNS) in a patient, the systems comprising:
at least one miniature device provided herein; and
an external system configured to generate one or more magnetic fields to direct and/or manipulate the miniature device within the patient.

In another aspect, provided herein are methods for providing localized treatment at a target site in the central nervous system (CNS) of a patient, the methods comprising:
providing a system described herein;
introducing the miniature device into the patient at an entry location in the CNS;
operating the external system to remotely propel and navigate the miniature device to the target site; and
performing one or more mechanical actions by the miniature device to effect the treatment.

In another aspect, provided herein are methods of treating Dandy-Walker malformation (DWM) in the CNS of a patient in need thereof, the methods comprising:
providing a system described herein;
introducing the miniature device into the patient at an entry location in the CNS;
operating the external system to remotely propel and navigate the miniature device to a Dandy-Walker cyst; and
operating the miniature device to fenestrate the Dandy-Walker cyst with the blade assembly to effect treatment.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 depicts manifestations of the Dandy-Walker Malformation;

FIG. 2 schematically illustrates an example of a system according to the subject matter disclosed herein;

FIG. 3 schematically illustrates an example of an External System according to the subject matter disclosed herein;

FIG. 4 depicts an example of a miniature device according to embodiments disclosed herein;

FIGS. 5A-5B illustrate perspective views of embodiments of the head of the miniature device illustrated in FIG. 4;

FIGS. 6A-6B illustrate overhead views of embodiments of the head of the miniature device illustrated in FIG. 4;

FIGS. 7A-7B illustrate side views of embodiments of the blade of the miniature device illustrated in FIG. 4;

FIG. 8 illustrates an overhead view of an embodiment of the blade of the miniature device illustrated in FIG. 4;

FIGS. 9A-9B illustrate the miniature device illustrated in FIG. 4 piercing a wall;

FIG. 10 illustrates an alternative view of the miniature device illustrated in FIG. 4 piercing a wall;

FIG. 11 depicts an example of a miniature device with a bore for fluids to drain from one side of a wall to the other;

FIG. 12 illustrates a perspective view of an embodiment of the miniature device illustrated in FIG. 4;

FIG. 13 illustrates a miniature device piercing a wall according to alternative embodiments disclosed herein;

FIG. 14 illustrates a cyst and its surrounding region of the CNS;

FIG. 15 illustrates an introducer according to embodiments disclosed herein;

FIG. 16 illustrates a retriever according to embodiments disclosed herein; and

FIGS. 17A-17N depicts a procedure treating a Dandy Walker cyst using systems and devices provided herein.

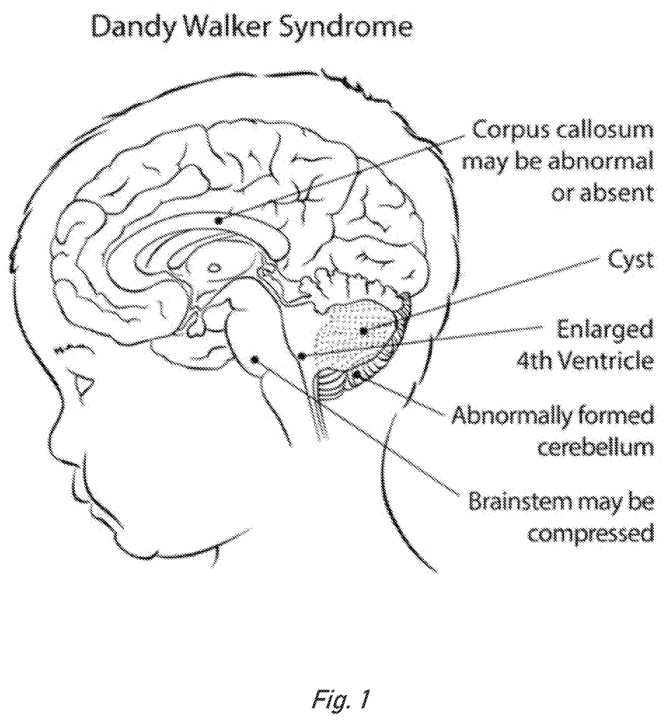

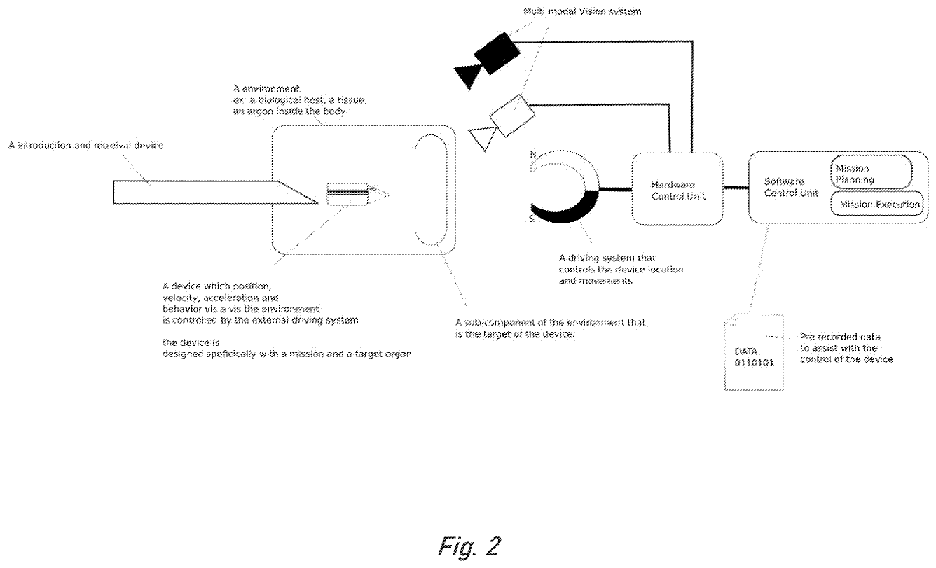

### DETAILED DESCRIPTION

In one aspect, provided herein are devices and systems that comprise a millimeter size tetherless object controlled or manipulated remotely by an external magnetic field, referred to herein as the “miniature device”, and separate from the miniature device an interactive hardware-software platform (referred to herein as the “External System” or “ES”) that generates, modulates and controls magnetic fields in a defined three-dimensional operational volume to propel and navigate the miniature device to a specific anatomical target to complete a (microsurgical) mission or task.

The miniature device is propelled and navigated remotely by the ES through specific anatomical milieu, exemplified by the lumen, cavity, vessel, tissue(s), and circuitry. The milieu can include homogeneous or heterogeneous components, such as lumens, respective luminal lining, and adjacent tissue(s). One such heterogeneous compartment is the Central Nervous System (CNS).

The miniature device may travel within the CNS to perform specific (micro) surgical, diagnostic or therapeutic mission(s).

The miniature device may be navigated through healthy or pathological domain(s) and/or combinations thereof. One such representative example is the non-communicating or obstructive hydrocephalus. An additional representative example is a Dandy Walker Malformation featuring an occipital cyst. The term “cyst” refers to a closed sac with anatomically and topologically defined boundaries comprised of specific tissue and membrane (wall).

The miniature device is introduced into the milieu via an introduction tool following a noninvasive or minimally invasive protocol as exemplified, but not limited to, intranasal or Touhy needle-mediated lower occipital delivery. A representative example of one such entry point is the foramen magnum. Another example of such an entry point is the lower lumbar spine.

A microsurgical, diagnostic or therapeutic mission or task comprises one or more actions, to be accomplished in the relevant anatomical milieu.

A mission or task includes a) insertion of the miniature device to an starting location in the CNS, exemplified but not limited to supra- or subarachnoid space including ventricles and cisterns; b) propulsion or travel to an anatomically determined locus or loci, c) microsurgical, diagnostic or therapeutic operation(s) or action(s) performed at the locus or loci of therapeutic interest, d) retrieval of the miniature device and its collection at a specified retrieval location.

One example of such microsurgical action is fenestration (puncture) of a Dandy Walker cyst in the lower occipital area to balance intracranial pressure in the CNS. This microsurgical action could be performed as one puncture or, if necessary, a series of punctures to achieve the desired therapeutic effect. The miniature device may perform the fenestration at a single locus or multiple loci on the cyst membrane or wall.

### The Mission

A mission comprises: (a) travel of the miniature device 101 to a target location inside a milieu described herein; and (b) a specific, safe and efficient mechanical activity performed by the miniature device at the target location. The activity could be performed either once or multiple times to achieve a desired therapeutic effect. The activity could be performed at a single or multiple pre-determined anatomical loci.

Travel needs both control and a set of specific actions mediated by an external system (ES) 801 described herein. In some embodiments, travel is performed in a first volume filled with media or liquid as exemplified by, but not limited to, cerebrospinal fluid (CSF). In some embodiments, the first volume is the negative space that exists between the brain matter and the skull. In other embodiments, two adjacent anatomical volumes or lumens are separated by a normal or pathological wall or membrane as exemplified by, but not limited to, a Dandy Walker cyst. The topology of the cyst could be concave, convex, or a combination thereof. In some embodiments, the wall is as thin as 5 μm and as thick as 5,000 μm.

For example, the bounding wall 501 of the cyst to be pierced is 10 to 500 μm thick, e.g., 50 μm thick. The cyst wall may have an elastic modulus of 1 to 100 MPa (e.g., 2 MPa) and a tensile strength 0.5 to 7 MPa (e.g., 1 MPa). The force needed to fenestrate the cyst wall may be 1 to 100 mN (e.g., 10 mN). The pressure at the tip for fenestration may be 1 to 200 mPa (e.g., 10 mPa).

In some embodiments, the path traveled by the miniature device is linear. In other embodiments, the three-dimensional path traveled by the miniature device is curvi-linear. In some embodiments, the distance traveled by the miniature device is 1 to 20 mm (e.g., 5 mm). In some embodiments, none of the miniature device remains behind in the subject after a procedure. In other embodiments, part of the miniature device is left behind in the subject after a procedure. In some embodiments, the miniature device pierces the cyst wall once. In some embodiments, the miniature device pierces the cyst wall multiple times. In some embodiments, the miniature device pierces the cyst wall between one and ten times. In some embodiments, the width of the fenestration (or each fenestration for multiple piercings) is 0.5 to 5 mm, for example 1 mm.

The mechanical activity can be performed for surgical reasons. The activity has an expected immediate effect from which secondary effects may also be expected. For example, the primary effect may be to drill a hole (fenestrate) in the wall 501 and the secondary effect may be to allow fluid to flow from one volume to the other volume through the hole in the wall. The overall therapeutic effect of this procedure is balancing intracranial pressure. The therapeutic effect of this microsurgery may provide both immediate as well as long-term therapeutic benefit(s) to the DWM patient(s) including but not limited to developmental, neurological, behavioral, performance, quality of life milestones.

The External System

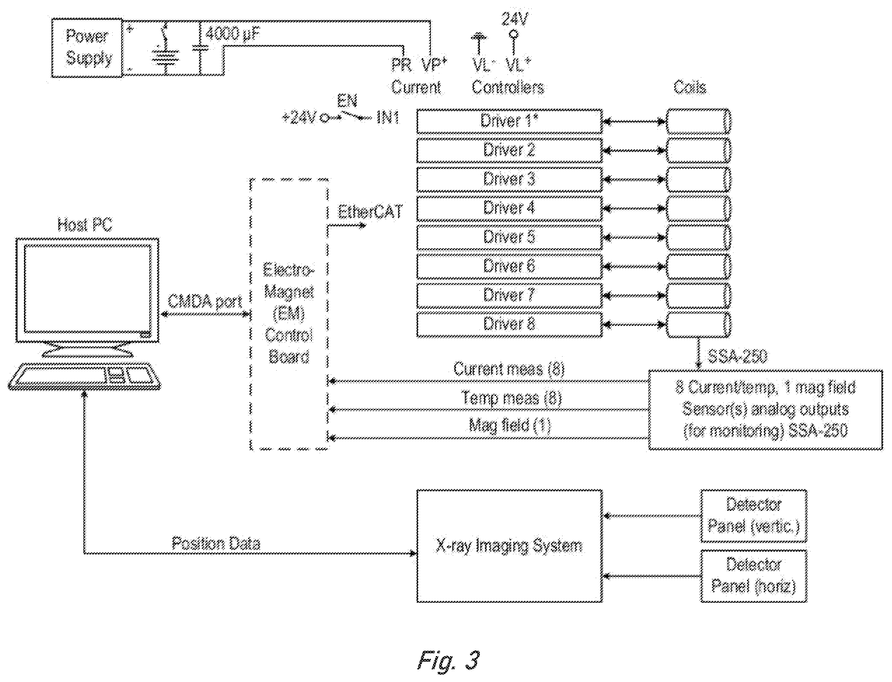

The External System 801 (ES) includes both a software module and a hardware system to achieve its function. An example of an External System (ES) is depicted schematically in FIG. 3. The ES uses magnetic fields to exert mechanical forces on the miniature device and to control it to perform specific actions. The forces, controllably and predictably exerted on the miniature device 101, are expected to have various consequences on the milieu. In some embodiments, the ES mediates propulsion and navigation of the miniature device from one predetermined position to another. For example, the ES controls specific miniature device motion(s) including, but not limited to, standalone axial or diametral spinning, rotation, vibration, tumbling, crawling, rocking or combined with lateral motion to drill or fenestrate through an obstacle exemplified by a biological wall/membrane. The foregoing action could be performed once or multiple times. The foregoing action could be performed in a single or multiple loci to achieve the desired effect. For example, specific motion around a certain point accomplishes a desired primary effect. In another example, the forces coax the miniature device to remain immobile near a predetermined position.

In some embodiments, the External System generates magnetic fields using Permanent Magnets (PM). In some embodiments, the Permanent magnets are mounted on a mechanical setup that holds the magnet and can move in three dimensions. In some embodiments, the permanent magnets can rotate. In some embodiments, there is only one permanent magnet. In other embodiments, there are several magnets that are actuated independently.

In some embodiments, the External System generates magnetic fields using Electromagnets (EM). An electromagnet comprises one, two, three or multiple coils. An electromagnet can, in addition, comprise a bobbin to support the coil and a yoke that modifies the electromagnetic properties of the coil. In some embodiments, the electromagnets are mounted on a mechanical setup that holds them at a fixed location with respect to the milieu where the miniature device resides. In some embodiments, the electromagnets are mounted on a mechanical setup that controllably and predictably determines the position of the electromagnets with respect to the milieu where the miniature device resides. In some embodiments, there is only one electromagnet. In some embodiments, there are electromagnets that are actuated according to predetermined control algorithms that take into the account the position, velocity, acceleration and pose of the miniature device extracted using separate algorithms that use X-ray images of the miniature device in real time. In some embodiments, the fields are generated in such a fashion that the forces applied to the miniature device cause it to rotate about its axis.

In some embodiments, the External System generates magnetic fields using a combination of Electromagnets (EM) and Permanent Magnets (PM). In some embodiments, both EM and PM are used in concert to generate the fields and applies forces to the miniature device.

In some embodiments, the External System generates the magnetic fields using six to twelve electromagnetic coils. For example, eight electromagnetic coils are used to generate the magnetic fields. Each coil is between 4 inches×4 inches×4 inches and 12 inches×12 inches×24 inches in size, for example, each electromagnetic coil is 8 inches×8 inches×15 inches in size. Each coil can carry up to 100 Amps, and for example runs at 30 Amps. In some embodiments, the electromagnetic coils have a ferromagnetic yoke. In other embodiments, the electromagnetic coils have no yoke. In some embodiments, the electromagnetic coils are arranged around the head of the subject in use. In some embodiments, the electromagnetic coils are between 5 to 25 cm, e.g., 15 cm, from the milieu.

The External System (ES) may use a variety of visualization or imaging systems to assist with the application of the forces and result in adequate control of the miniature device. In some embodiments, the ES uses X-rays to image the miniature device inside the targeted milieu. In some embodiments, the ES uses two X-rays applied in a stereovision system to image the miniature device inside the targeted milieu to determine its three-dimensional position with respect to landmarks or fiducial markers used as a reference. In some embodiments, the ES uses optical stereovision to determine the position of the EM or PM with respect to fiducial markers or landmarks used as a reference. In some embodiments, the ES uses a combination of X-ray stereovision and optical stereovision to monitor the position of the miniature device, only visible under X-ray vision, with respect to the external EM or PM, only visible under optical vision.

The External System (ES) software module comprises: a planning software submodule and a hardware-control software submodule. In some embodiments, the ES software module makes use of pre-recorded digital data, such as MRI scans, CT scans, etc. . . . to assist with the activities performed by both submodules. In some embodiments, MRI scans are analyzed by the software module and digital three-dimensional objects representing various tissue masses present in the host environment are generated automatically. In some embodiments, the hardware-control software module uses digital information from the optical or X-ray stereovision system and computes automatically the mathematical transformation to allow display, in a single referential, three-dimensional data generated from MRI, vision and X-ray system.

### The Miniature Device

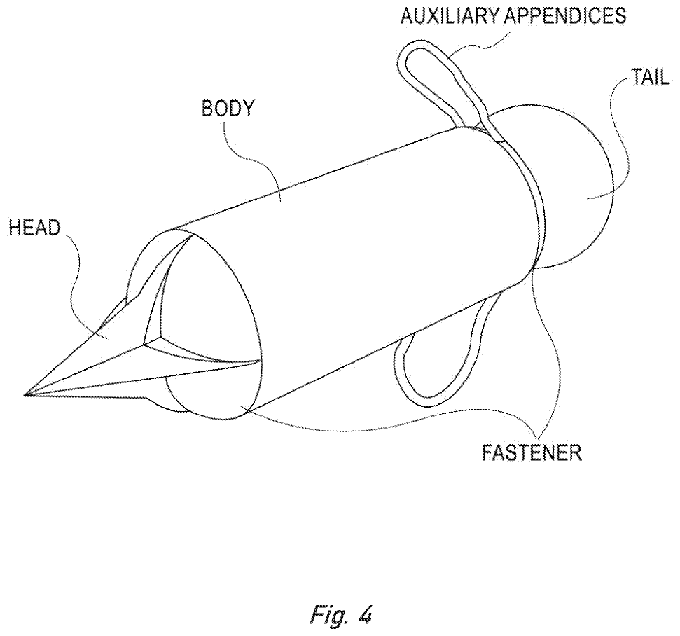

An exemplary miniature device 101 is depicted in FIG. 4. The miniature device is a micro-object with 50-5,000 μm dimensions comprising: (a) a body; (b) a head; (c) a tail, and optionally one or more auxiliary appendices.

The miniature device's dimensions, geometry, etc., are adapted to facilitate performance of its mission within the milieu. In some embodiments, the miniature device is elongated in one dimension. In some embodiments, the miniature device has a total length between 1 and 20 mm, e.g., 7 mm. In some embodiments, the miniature device has an outer diameter between 1 and 5 mm, e.g., 2.5 mm. In some embodiments, the miniature device has a total length between 50 and 10,000 microns. In some embodiments, the miniature device has an outer diameter between 50 and 5,000 microns.

In some embodiments, the miniature device body 301 is a cylinder, a cone or a trapezoid. In other embodiments, the body of the miniature device is a sphere or spheroid. In some embodiments, the body is made of a permanent magnet magnetized along the long axis of the miniature device. In some embodiments, the permanent magnet is magnetized along a direction that is substantially perpendicular to the long axis of the miniature device. In some embodiments, the body is made of an X-ray opaque material. In some embodiments, the body is made of a rare earth magnet, coated with electroplated nickel and gold, ceramic, plastic or other biocompatible material substantially non-magnetic.

In some embodiments, the body of the miniature device is made from a neodymium magnet. In some embodiments, the grade of the neodymium magnet is from N40 to N55, e.g., N50. In some embodiments, the magnet's residual induction (Br) is from 12 to 15 KG, e.g., 14 KG. In some embodiments, BHmax is between 38 and 56 MGOe, e.g., 47 MGOe. In some embodiments, the magnet's intrinsic coercivity, (Hci) is greater than 11 Koe. In some embodiments, the magnet's intrinsic normal coercivity, (HcB) is greater than 10 Koe. In some embodiments, the magnet's maximum operating temperature is 60 to 80° C, preferably 80° C.

In some embodiments, the miniature device head 201 comprises a single metallic blade 203. In some embodiments, the blade is made of stainless steel. In some embodiments, the blade is made of a polymeric material or a ceramic material. In some embodiments, the blade has a minimum yield strength of 200 to 2,000 Mpa, preferably >500 Mpa. In some embodiments, the blade thickness is between 10 and 500 μm, e.g., 100 μm. In some embodiments, the blade forms an angle of 15 to 90 degrees, e.g., 45 degrees. In some embodiments, the blade length is between 0.5 and 10 mm, e.g., 1 mm. In some embodiments, the blade mass is between 0.5 and 10 mg, e.g., 2 mg. In some embodiments, the blade tip 205, most distal point of the miniature device, has a radius of curvature at its apex of between 1 and 10 μm, e.g., 5 μm. In some embodiments, the blade tip size 10 μm from the apex is between 1 and 20 μm, e.g., 10 μm.

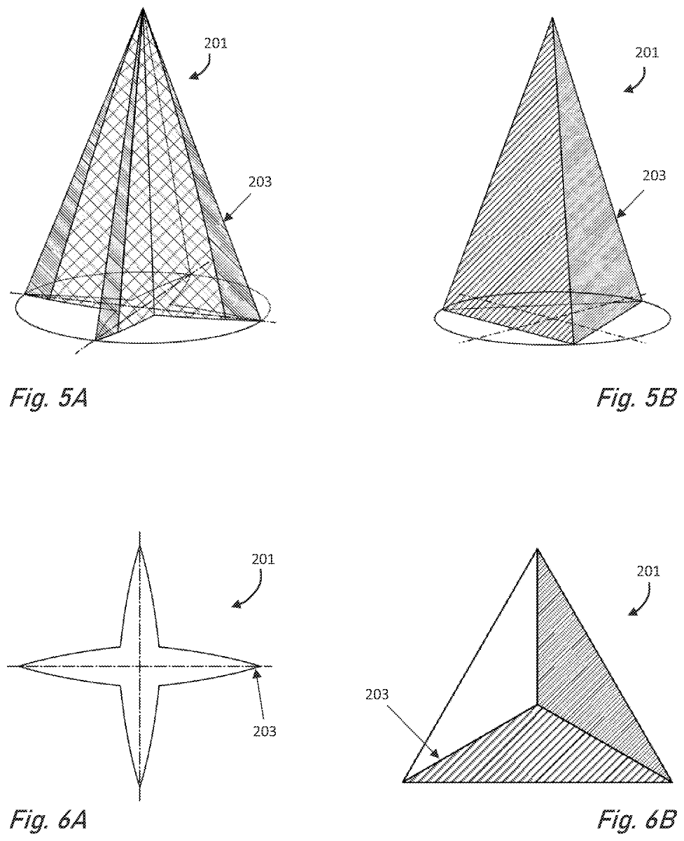

In some embodiments, the miniature device head comprises several metallic blades 203 made of stainless steel and arranged in a concentric pattern (FIGS. 5A, 6A). In some embodiments, the head is a pyramid or a tetrahedron (FIGS. 5B, 6B). In some embodiments, the blade width is substantially equal to the body diameter. In some embodiments, the blade width is greater than the body diameter. In some embodiments, the blade width is lesser than the body diameter. In some embodiments, the blade length is between 1 and 10 mm. In some embodiments, blade thickness is between 10 μm and 1 mm.

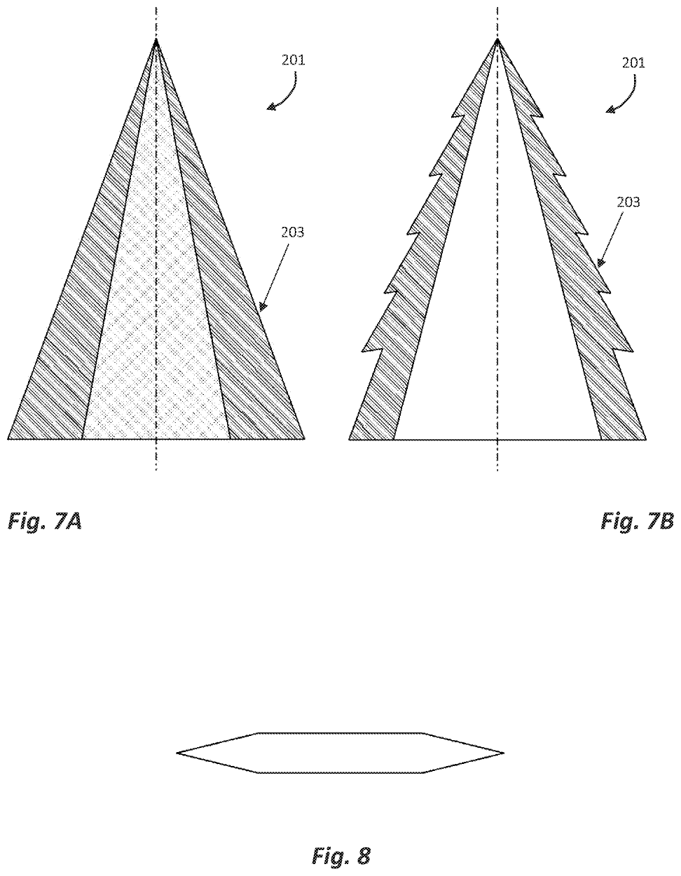

In some embodiments, the blade 203 has one of more smooth cutting edges (FIG. 7A). In some embodiments, the blade has small serrations or teeth along the cutting edge (FIG. 7B). In some embodiments, the serrations are 10 to 100 μm long. In some embodiments, the blade tip 205, most distal point of the miniature device, has a radius of curvature between 0.5 micrometers and 50 micrometers. In some embodiments, the blade 203 is attached to the body 301 using medical grade adhesive 303. In some embodiments, the blade is spot welded to the body and medical grade adhesive is used to re-enforce the affixation of the blade to the body. In some embodiments, the tail of the miniature device is shaped to provide a distinct and recognizable signal when observed using X-ray under various angles.

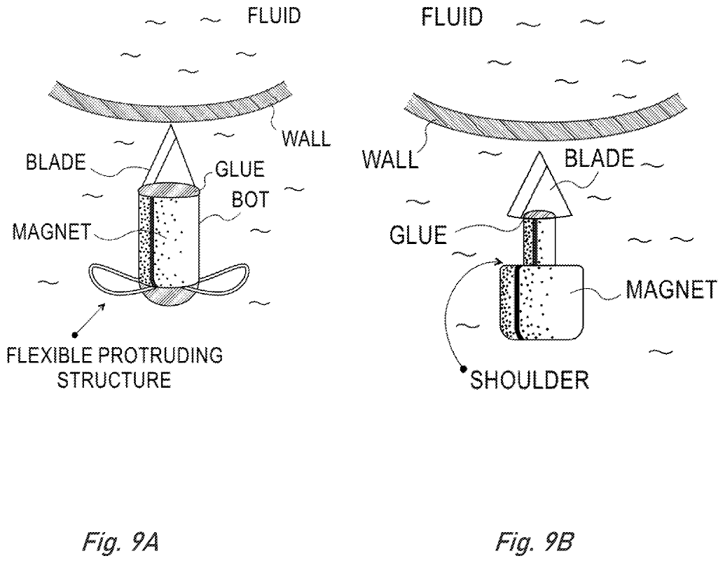

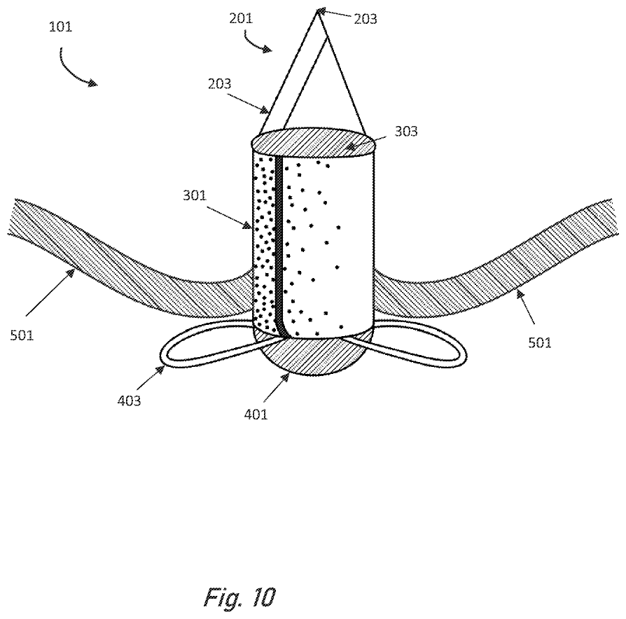

In some embodiments, a flexible protruding structure 403 is attached to the body 301 (FIG. 9A).

In some embodiments, the body 301 has a variable diameter along its length. For example, the diameter may change abruptly, creating a circumferential shoulder (FIG. 9B). In some embodiments, the shoulder or flexible protruding structure provides means to stop progression of the miniature device. For example, the miniature device drills into a wall 501 and the shoulder or flexible protruding structure prevents it from progressing past the wall entirely. The miniature device operation can be performed on a convex or concave (micro) surface of the cyst to achieve the best therapeutic effect.

In some embodiments, the shoulder or flexible protruding structure helps apply forces to the wall 501 while the body 301 experiences forces tending to pull the miniature device to traverse the wall. In this case, the forces on and experienced by the wall lead to the wall moving. In some embodiments, wall movement leads to forcing the liquid past the wall to ebb or flow through the hole in the wall around the body of the miniature device.

In some embodiments, the miniature device is driven with a static unidirectional force, arising from the interaction between the external magnetic field generated by the External System (ES) 801 and a permanent magnet that forms part of the miniature device.

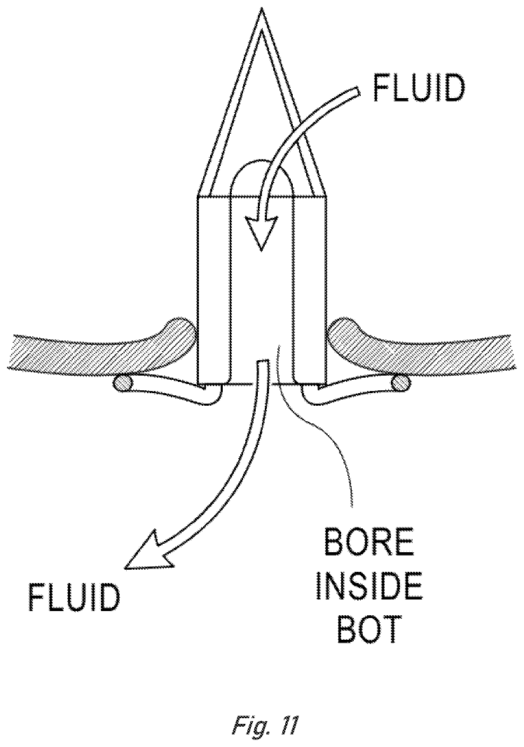

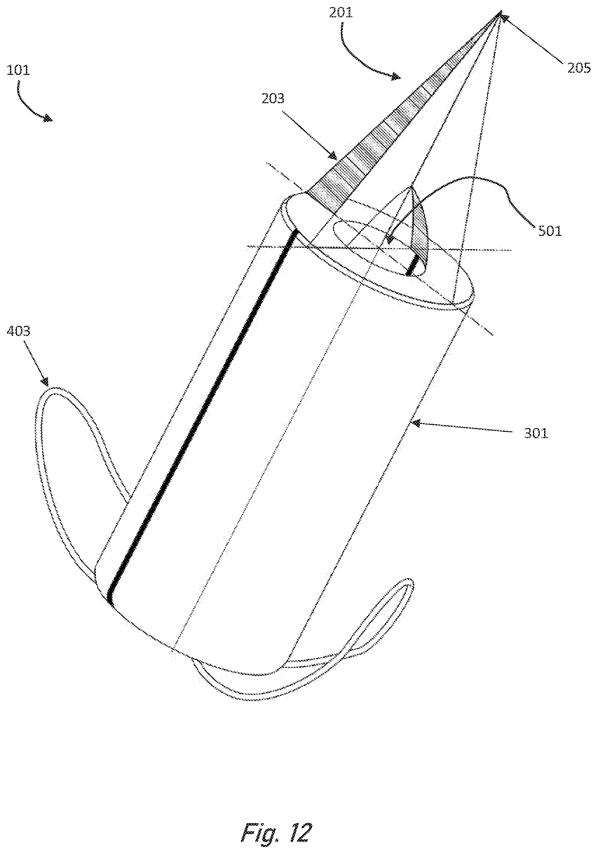

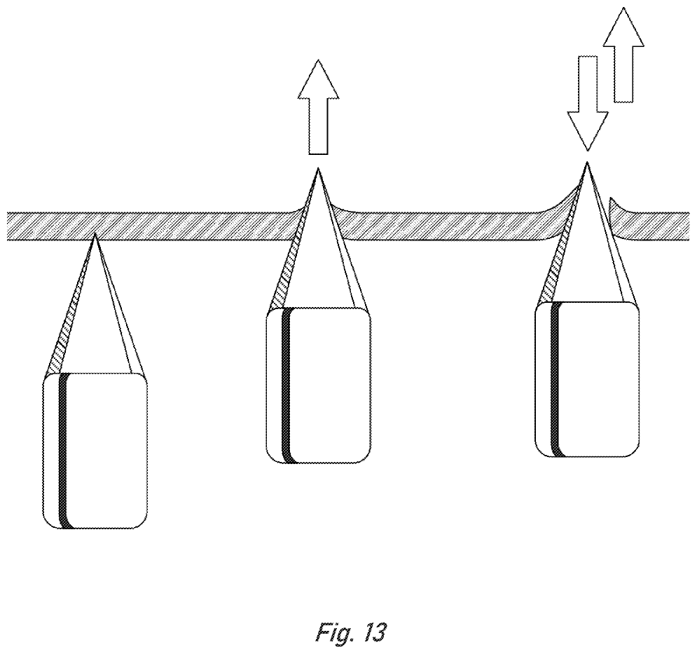

In the embodiment depicted in FIGS. 11-12, a bore 501 formed inside the miniature device body provides means for fluids to drain from one side of the wall to the other. In some embodiments, the force required to pierce the wall is between 0.1 mN and 100 mN. In some embodiments, the magnetic field amplitude that the miniature device is subjected to is between 0.1 mT and 1000 mT. In some embodiments, the magnetic field gradient that the miniature device is subjected to is between 0.1 mT and 1000 mT.

In the embodiment depicted in FIG. 13, the miniature device is driven with a variable force coaxing the miniature device to drive back-and-forth while piercing the membrane. In some embodiments, the frequency of the back-and-forth motion is between 0.1 and 100 Hertz. The motion could be rotation, rocking, edging, cutting, drilling or a combination thereof. In one embodiment, one particular frequency of the motion contributes to lowering the force threshold for piercing the wall.

### The Milieu

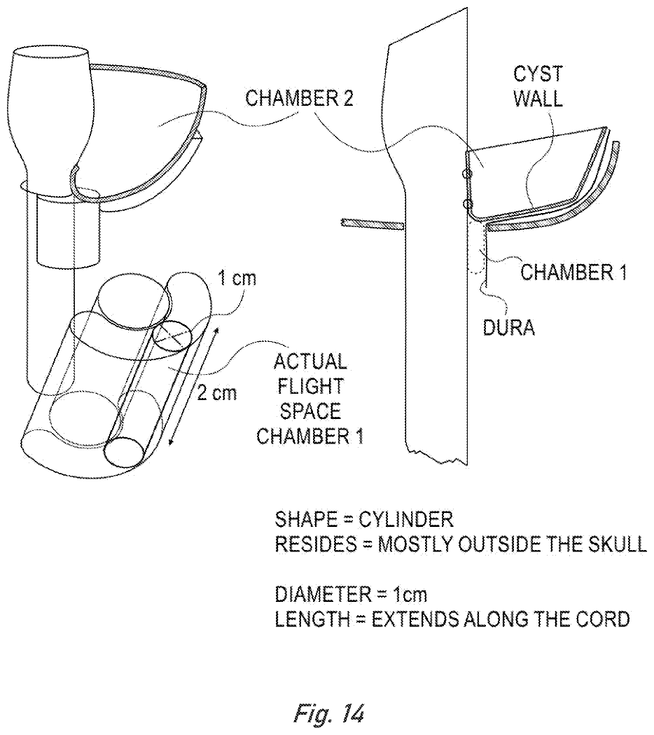

In some embodiments, the milieu is inside the human body. In some embodiments, the milieu or media is within the skull. In some embodiments, the milieu or media extends outside the skull and into the subarachnoid space around the spinal cord. In some embodiments, the miniature device is delivered using a delivery or introduction device 601 in a first volume (CHAMBER 1 in FIG. 14). In some embodiments, the first volume extends inside the subarachnoid space outside the skull. In some embodiments, the first volume extends through the foramen magnum, inside the skull in a region of the skull that is typically the location of the cisterna magna. In some embodiments, the miniature device mission is to drill or perforate a boundary, also referred to as the cyst wall, and penetrate a secondary fluid filled volume (CHAMBER 2 in FIG. 14), referred to as the cyst.

### The Introduction and Retrieval Tool Kit

The miniature device introduction and retrieval tool kit comprises a sharp rigid pointed surgical instrument fitted with a cannula. In some embodiments, the cannula is rigid and made of titanium or other non-magnetic metal or plastic. In some embodiments, the cannula is flexible. In some embodiments, the cannula outer diameter ranges from 1 mm to 10 mm. In some embodiments, the outer diameter ranges from 1 mm to 5 mm, for example, 3.5 mm. In some embodiments, the cannula is stabilized by a mechanical arm. In some embodiments, the cannula is stabilized by hand. In some embodiments, the cannula is automatically or robotically stabilized. In some embodiments, the cannula is be guided using X-rays. In some embodiments, the cannula is guided using X-rays. In some embodiments, the cannula is guided stereotactically.

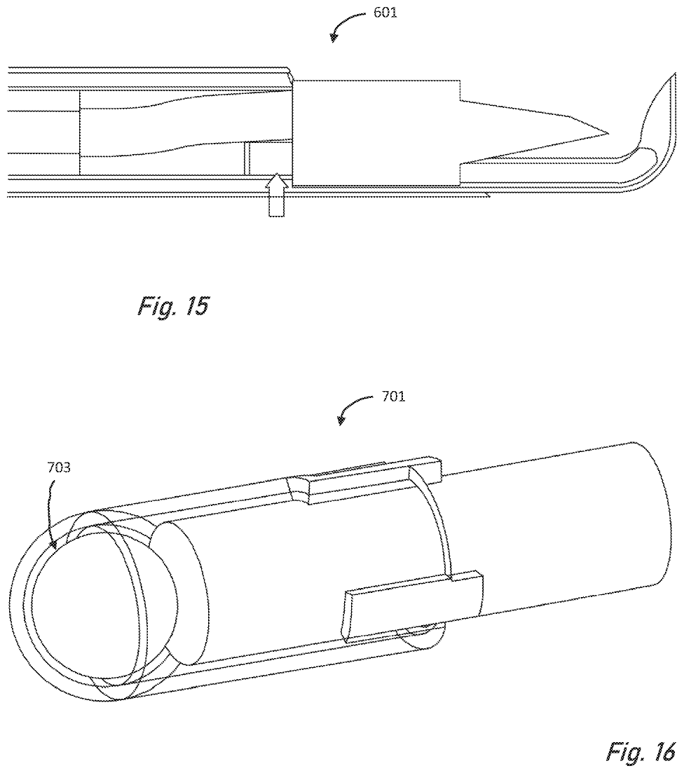

In some embodiments, the introduction and retrieval tool kit also comprises a separate, interchangeable miniature device holder (the “introducer”) 601 that is used to insert the miniature device into the subject and to release it into, e.g., the cisterna magna. In some embodiments, the introducer is disposable and/or configured for a single use. In some embodiments, the introducer is pre-loaded at its distal end with the miniature device. The miniature device may be held in place by a small (e.g., a 0.75 mm cylindrical) magnet at the distal end of the introducer. As depicted in FIG. 15 the small cylindrical magnet (pointed to by the arrow) sits on the bottom of the introducer and holds the miniature device in place, to allow for controlled orientation of it towards the cyst before launch.

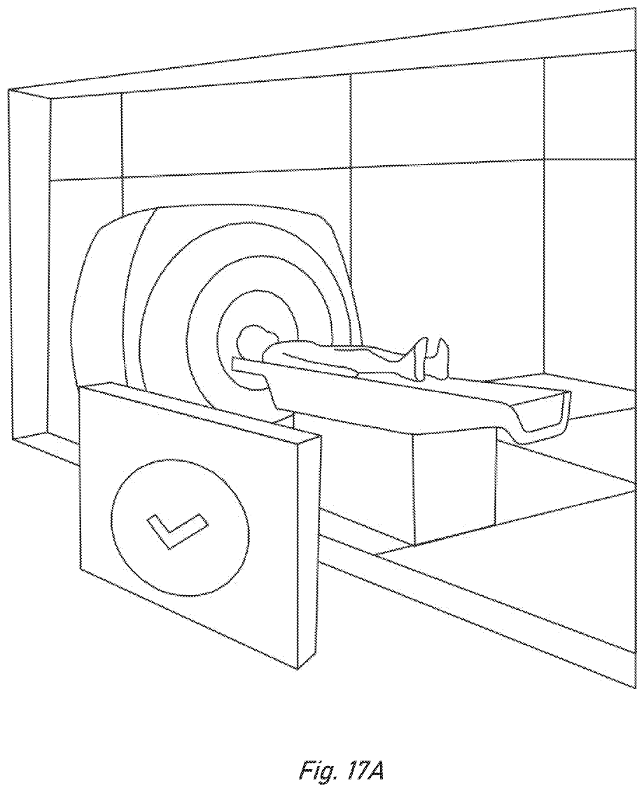

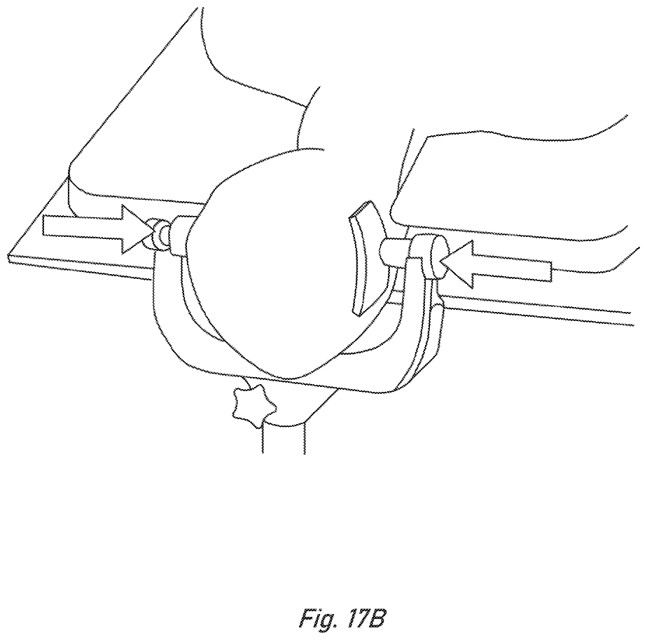

In some embodiments, the introduction and retrieval tool kit also comprises a separate, interchangeable miniature device retriever (the “retriever”) 701, which can replace the introducer in the cannula and is used to retrieve the miniature device from the subject after fenestration of the cyst. In some embodiments, the retriever is disposable and/or configured for a single use. In some embodiments, the retriever has a tip with a magnet 703 at its distal end that is used to attract and capture the miniature device and remove it from the subject. As depicted in FIG. 16, the tip of the retriever has a cage containing a small (e.g., 2 mm) spherical magnet that can freely rotate, to allow for the controlled orientation of the miniature device during retrieval from the subject.

In some embodiments, the introducer is the same as the retriever. In some embodiments, the retriever is a separate tool that comprises a net to catch the miniature device and a Magnet at the end of a wire to attract the miniature device towards the net. In some embodiments, the ES system simply drives the miniature device back to the net and the net is mechanically actuated using tethers to close around the miniature device.

In some embodiments, a sharp rigid pointed surgical instrument fitted with a cannula enters the body in the neck area and is pushed through the soft tissue in the direction of the foramen magnum. In some embodiments, the tip of the sharp rigid pointed surgical instrument fitted with a cannula is advanced until it reaches the region outside the skull near the foramen magnum. In some embodiments, the tip of the sharp rigid pointed surgical instrument fitted with a cannula is advanced until it reaches the region inside the skull near the foramen magnum. This region can be the space referred to as the cisterna magna.

In some embodiments, the External System (ES) 801 monitors the position of the sharp rigid pointed surgical instrument fitted with a cannula using a stereo vision camera. In some embodiments, the route of the sharp rigid pointed surgical instrument fitted with a cannula is planned in advance by medical personnel using the planning submodule. In some embodiments, the route is charted in the planning submodule using MRI data. In some embodiments, the position of the sharp rigid pointed surgical instrument fitted with a cannula is represented, as perceived by an optical stereovision system, in real time, on an electronic display station.

In some embodiments, the proximal end of the cannula is equipped with a valve-like system through which the sharp rigid instrument can be removed while preventing fluids from inside the body to drain out. In some embodiments, the sharp instrument is removed to make way for the miniature device to travel up and down the cannula. In some embodiments, the miniature device is placed inside the cannula, through the valve-like system and coaxed to travel all the way to the tip of the cannula by flushing a fluid. In some embodiments, a miniature device holder that fits inside the cannula and that holds the miniature device on its distal end is inserted into the cannula. In some embodiments, the miniature device holder is longer than the cannula. In some embodiments, the miniature device holder distal end is advanced further, until the miniature device is fully out of the cannula. In some embodiments, the miniature device holder can be coaxed to release its hold on the miniature device on demand using mechanical levers or release wires available on the proximal end.

In some embodiments, the action of releasing the hold on the miniature device is monitored by the ES. In some embodiments, the action of releasing the hold on the miniature device is synchronized with the ES. In some embodiments, the ES uses stereo X-ray vision system to evaluate the position of the miniature device. In some embodiments, the ES starts generating magnetic fields of adequate intensity and characteristic as the miniature device is being released from the miniature device holder.

One or more components of the system may be provided, mutatis mutandis, as described in any one or more of WO2019/213368, WO2019/213362, WO2019/213389, WO2020/014420, WO2020/092781, WO2020/092750, WO2018/204687, WO2018/222339, WO2018/222340, WO2019/212594, WO2019/213368, WO2019/005293, WO2020/096855, WO2020/252033, WO2021/021800, WO2021/092076, WO2021/126905, WO2021/216463, WO2022/119816 and U.S. Provisional application Nos. 63/191,454, 63/191,418, 63/191,515, and 63/191,497, the full contents of which are incorporated herein by reference.

### EXAMPLES

### Use of a Tether-Free Robotic System to Remotely Treat Dandy-Walker Malformation (DWM)

This Example presents a platform or system, as well as a procedure for using it, for treating a Dandy-Walker malformation (DWM) This platform has four main components and accessories, namely:

A DWM Bionaut: an untethered particle/miniature device with a permanent magnet and blade, specifically designed to safely and reliably fenestrate a Dandy-Walker cyst.

A single use disposable introducer/retriever kit designed to insert the DWM Bionaut into the cisterna magna and retrieve it from the cisterna magna after cyst fenestration.

A Magnetic Propulsion System (MPS) with eight electromagnetic coils, driver and software modules that provide a magnetic field and propel the DWM Bionaut to an intended location. It includes various software components to plan and control the DWM Bionaut for insertion, navigation, therapeutic fenestration and return for retrieval, including:
A Planning Software Module: utilizing a pre-recorded MRI or intra-procedure Cone Beam CT (CBCT), the planning software recommends a path between entry location, penetration target, and retrieval location.
A Tracking Software Module: utilizing commercially available biplanar fluoroscopy to detect the DWM Bionaut in two orthogonal two-dimensional X-ray images and calculate the three-dimensional location of the DWM Bionaut in the images in real time.
An MPS Controller Software Module: a software module that activates the MPS to achieve navigation of the DWM Bionaut to and from the intended targeted location. The MPS controller software integrates path executions, a control loop algorithm, procedure recording, a user interface and safety controls.

A Patient Head Holder or skull clamp: a device to hold the patient's head in a pre-determined clinically acceptable position for the procedure and adapts to a neurology suite imaging table.

The system also uses a conventional X-ray system that integrates into a neuro-endovascular interventional fluoroscopy suite.

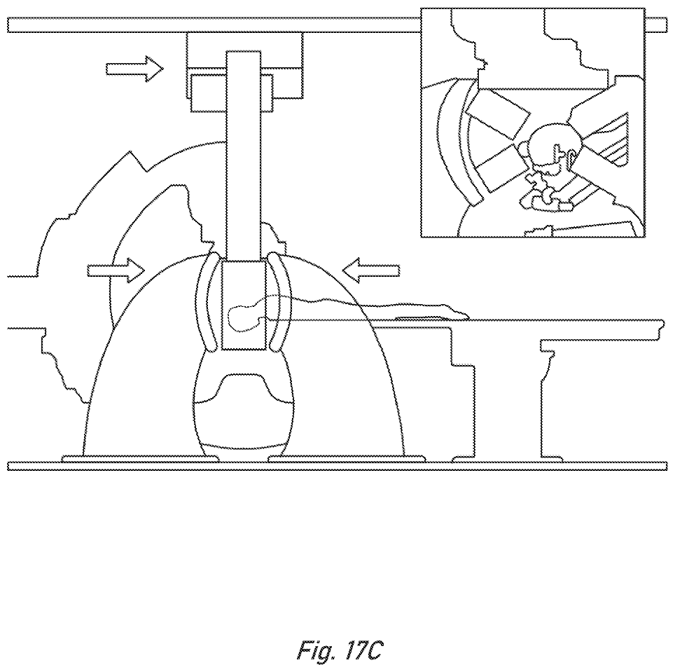

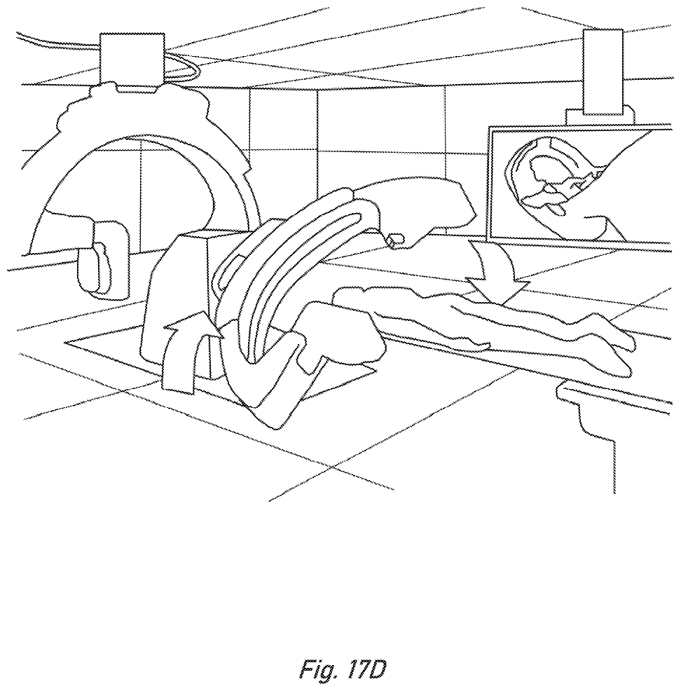

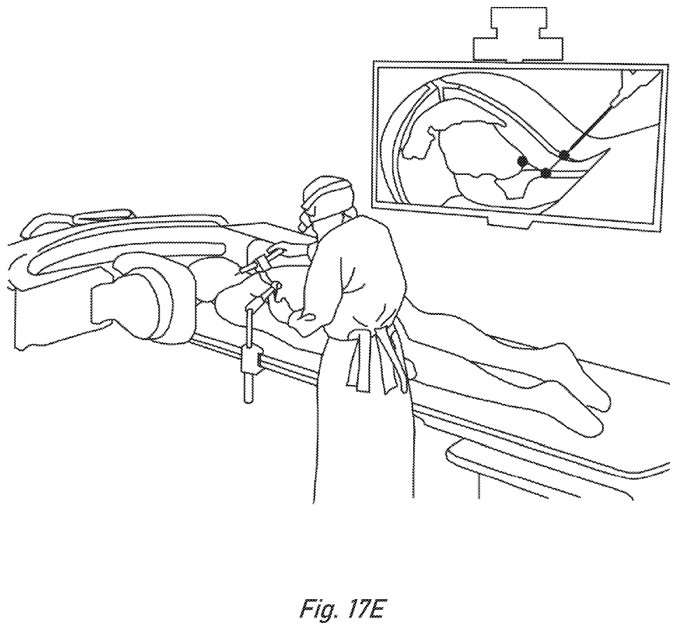

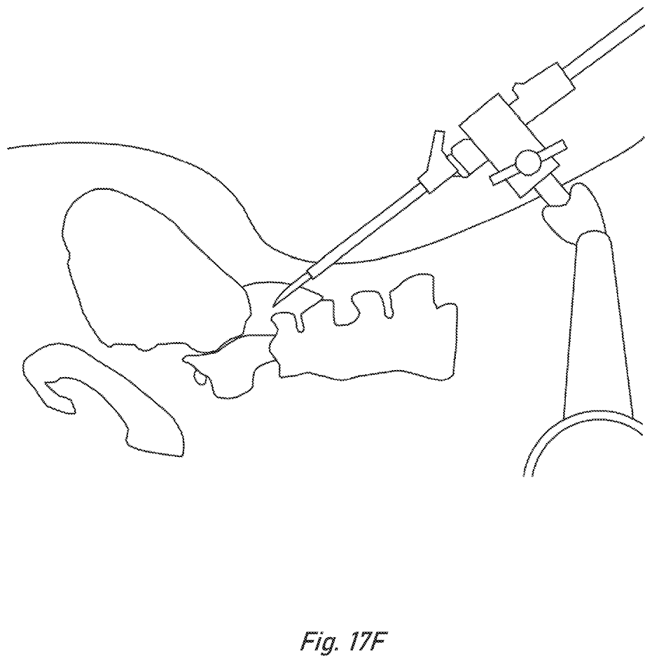

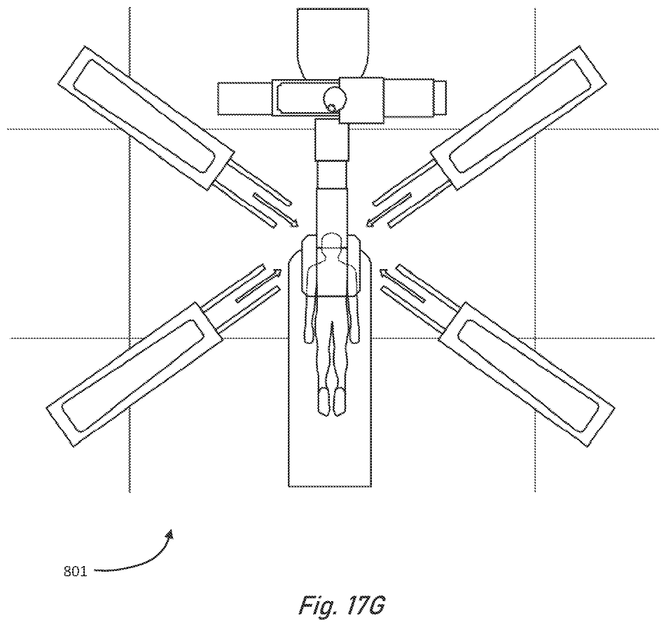

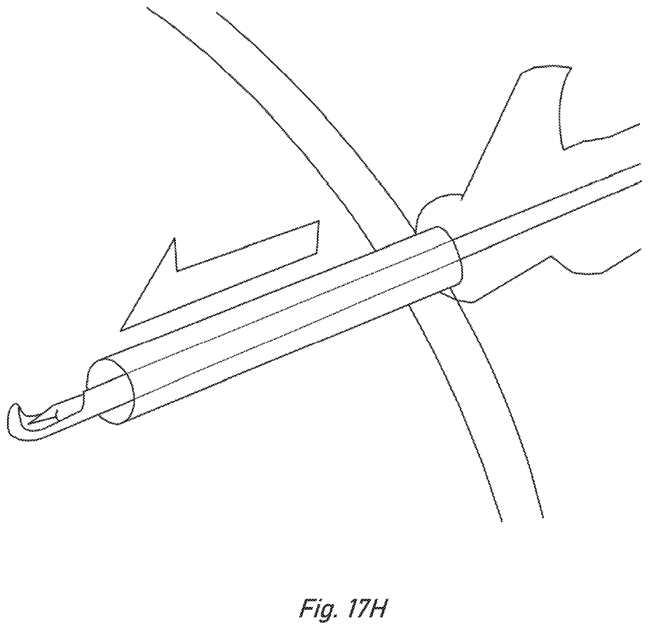

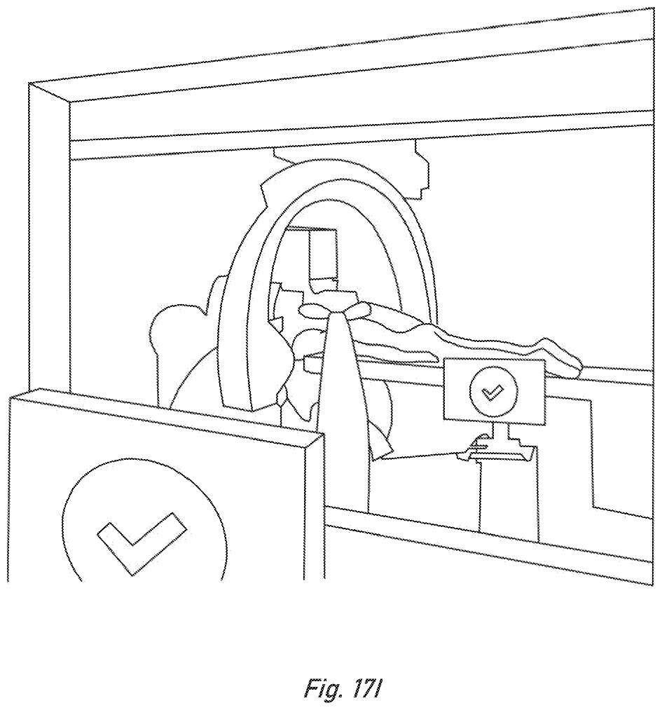

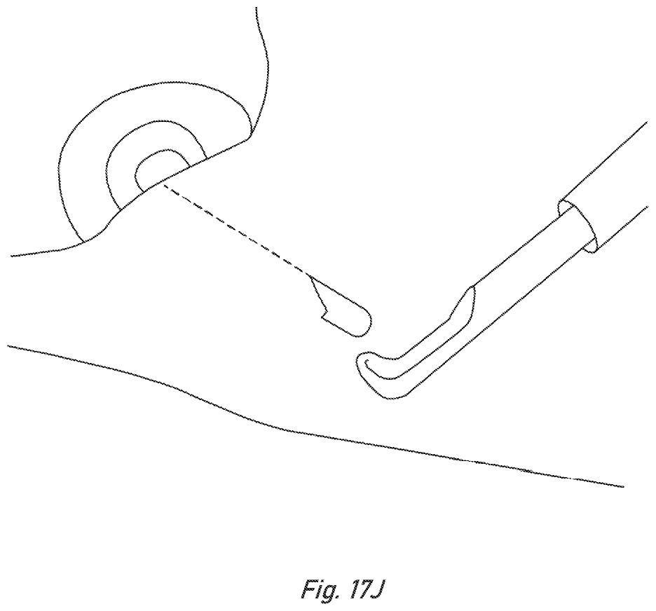

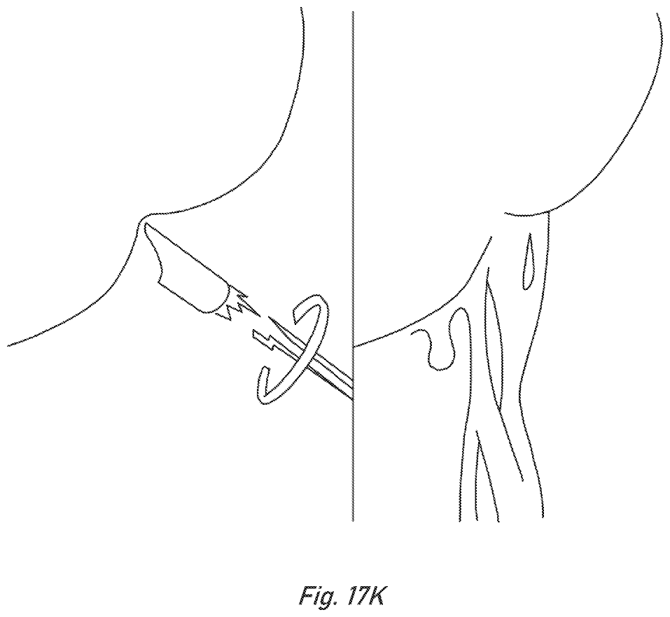

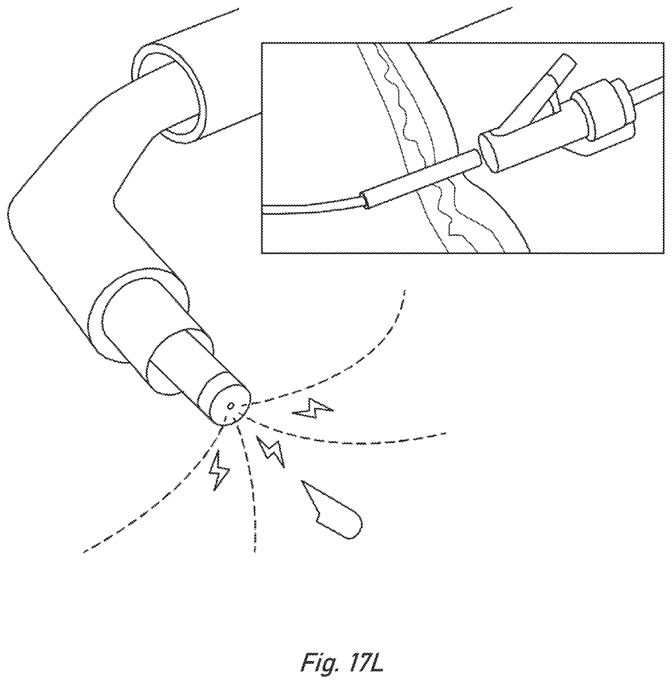

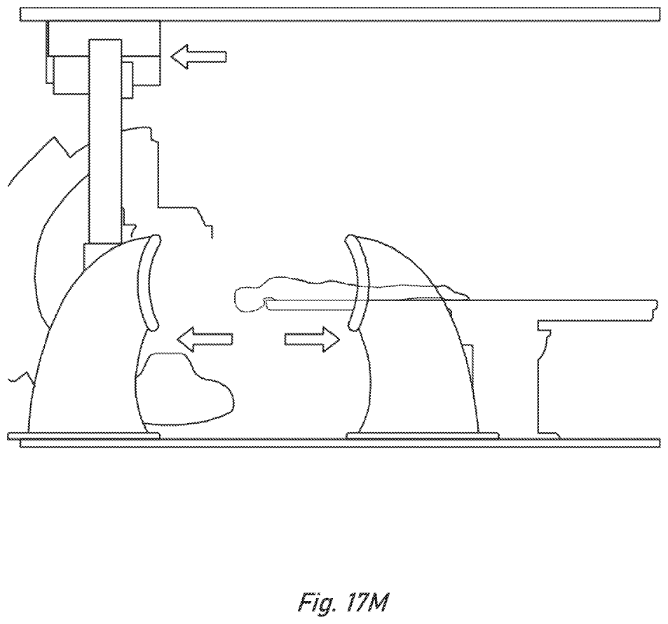

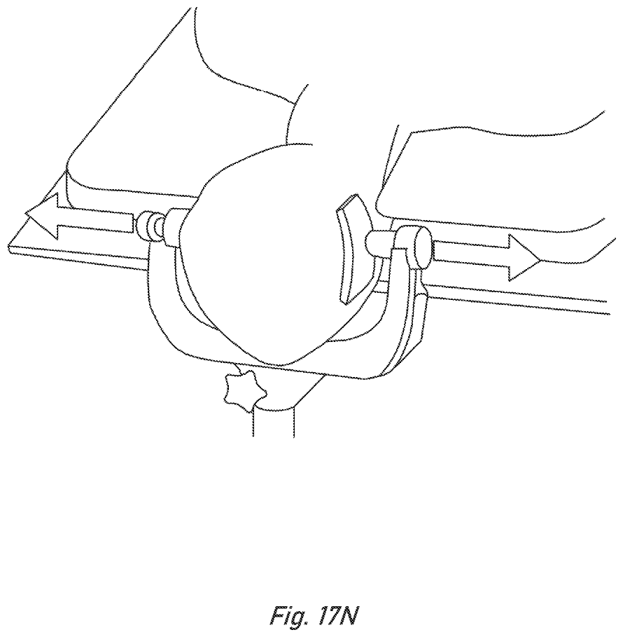

Prior to the procedure, the patient obtains an MRI to confirm the DWM diagnosis and to confirm the applicability of the platform to perform DWM microsurgery (FIG. 17A). The MRI is then used to plan a trajectory to the cyst, to determine a treatment plan and treatment parameters and to plan a retraction path from the cyst. The patient is brought into the operating room and placed in a prone position on a surgical bed (FIG. 17B). The patient's angled head is affixed using a skull clamp to prevent further movement of the head and neck. A fit check around the patient head is performed with both C-arms and the EM system (FIG. 17C). After the fit check the second C-arm and EM system is returned to their parked positions; with the frontal C-arm, a CBCT scan of the patient is performed (FIG. 17D). The MRI and CBCT scans are fused; a path for the trocar and the trajectory of the DWM Bionaut are determined and the MRI and CBCT scans are co-registered to live fluoroscopy. Under fluoroscopic guidance, the trocar is advanced along the planned trajectory to gain access to the cisterna magna (FIG. 17E). Once inside the cisterna magna, the cannula handle in the instrument holder is locked; the trocar is removed, and contrast is injected to visualize cisterna magna volume and cyst boundary (FIG. 17F). The second C-arm and EM system are moved from their parked positions and are positioned around the patient's head (FIG. 17G). Using the cannula as a passageway, the introducer loaded with the DWM Bionaut at the tip is inserted into the patient into the cisterna magna (FIG. 17H). The EM system is turned on to initiate launch of the DWM Bionaut off the introducer (FIG. 17I). The DWM Bionaut is oriented towards the cyst and magnetically guided and propelled along the planned trajectory to the target using the planning/operational software interface (FIG. 17J). Guided by the magnetic fields generated by the EM system, the DWM Bionaut fenestrates the cyst wall (FIG. 17K, left side), which allows fluid to flow in the cisterna magna, thereby restoring normal flow of CSF (FIG. 17K, right side). The introducer is replaced with the retriever; the DWM Bionaut is guided back (using magnetic fields generated by the EM system) towards the cannula and is captured by the retriever and removed through the cannula (FIG. 17L). The EM system is turned off and is returned (as well as the second C-arm) to parked position (FIG. 17M). A final CBCT scan is performed to confirm cyst fenestration. To end the procedure, the cannula is removed, the entry point is closed, and the skull clamp is removed from the patient's head (FIG. 17N).

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A miniature device configured to be directed by an external magnetic field along a path to a target site in the central nervous system (CNS) within a patient, and to perform one or more mechanical actions at the target site under manipulation by an external magnetic field, said miniature device comprising a body having a head portion and a tail portion defining a longitudinal axis spanning therebetween, said head portion comprising a blade assembly with one or more blades.

2. The device according to claim 1, the blade assembly comprising a plurality of blades arranged in a cruciform configuration perpendicular to the longitudinal axis.

3. The device according to claim 1, the blade assembly comprising a pyramidal member extending along the longitudinal axis, wherein lateral edges thereof constitute the blades of the blade assembly.

4. The device according to claim 1, wherein the blade assembly is wider than the body.

5. The device according to claim 1, wherein said blades are serrated.

6. The device according to claim 1, wherein said tail portion defines a shape distinct from that of the body.

7. The device according to claim 1, wherein the diameter of the body varies in the longitudinal direction.

8. The device according to claim 7, the body comprising a circumferential head-facing shoulder, wherein the diameter of the body between the head portion and the shoulder is less than the diameter of the body between the shoulder and the tail portion.

9. The device according to claim 7, wherein the diameter of the body gradually decreases between the tail portion and the head portion.

10. The device according to claim 1, further comprising one or more arresting members protruding laterally from the body.

11. The device according to claim 10, wherein the arresting members protrude from the body at or adjacent the tail portion.

12. The device according to claim 10, wherein the arresting members are made from a flexible material.

13. The device according to claim 1, wherein the body defines an elongate shape along the longitudinal axis.

14. The device according to claim 1, wherein the body comprises a magnetic material.

15. The device according to claim 14, the magnetic material being magnetized along the longitudinal axis.

16. The device according to claim 15, wherein the magnetic material is a neodymium magnet.

17. The device according to claim 16, wherein the neodymium magnet has a grade no less than N40 and no greater than N55.

18. The device according to claim 1, wherein the body has a diameter between 1 mm and 5 mm.

19. The device according to claim 1, wherein each blade has a minimum yield strength no lower than 200 MPa.

20. The device according to claim 19, wherein each blade has a minimum yield strength no lower than 500 MPa.

21. The device according to claim 1, the body being made of a radiopaque material.

22. The device according to claim 1, having a length along the longitudinal axis between 1 mm and 20 mm.

23. The device according to claim 1, having a length along the longitudinal axis between 1 mm and 20 mm, wherein the body defines an elongate shape along the longitudinal axis and has a diameter between 1 mm and 5 mm, wherein the body comprises a neodymium magnet magnetized along the longitudinal axis and having a grade no less than N40 and no greater than N55, and wherein the blade assembly comprises a single metallic blade having a minimum yield strength no lower than 500 MPa.

24. A system configured to facilitate treatment by microsurgery at a target site in the central nervous system (CNS) in a patient, the system comprising:
at least one miniature device according to claim 1; and
an external system configured to generate one or more magnetic fields to direct and/or manipulate the miniature device within the patient.

25. The system according to claim 24, wherein the external system is configured to direct the miniature device to move along path such that the head portion trails behind the body.

26. The system according to claim 24, wherein the external system comprises one or more electromagnets, one or more permanent magnets, or a combination thereof for generating the magnetic fields.

27. The system according to any claim 24, wherein the external system comprises a software module and a hardware system.

28. The system according to claim 27, wherein said software module comprises a planning software submodule configured to recommend a path between an entry location, the target site, and a retrieval location.

29. The system according to claim 27, wherein said software module comprises a controller software module configure to operate the external system to navigate the miniature device along a path to and from the targeted site.

30. The system according to claim 24, wherein said external system comprises a visualization system.

31. The system according to claim 24, wherein the visualization system uses X-ray stereovision, optical stereovision, or a combination thereof.

32. The system according to claim 24, further comprising a tool for introducing the miniature device into the CNS.

33. The system according to claim 24, further comprising a tool for retrieving the miniature device from the CNS.

34. A method for providing localized treatment at a target site in the central nervous system of a patient, the method comprising:
providing a system according to claim 27;
introducing said miniature device into the patient at an entry location in the CNS;
operating said external system to remotely propel and navigate said miniature device to the target site; and
performing one or more mechanical actions by said miniature device to effect the treatment.

35. The method according to claim 34, further comprising the step of retrieving the miniature device at a specified retrieval site.

36. The method according to claim 34, wherein the mechanical actions are performed at a single locus or at multiple loci at the target site.

37. The method according to claim 34, wherein the treatment is for Dandy-Walker malformation (DWM).

38. The method according to claim 34, wherein the one or more mechanical actions comprises operating the miniature device to fenestrate a Dandy-Walker cyst with the blade assembly

39. A method of treating Dandy-Walker malformation (DWM) in the central nervous system of a patient in need thereof, the method comprising:
providing a system according to claim 27;
introducing said miniature device into the patient at an entry location in the CNS;
operating said external system to remotely propel and navigate said miniature device to a Dandy-Walker cyst; and
operating the miniature device to fenestrate the Dandy-Walker cyst with the blade assembly to effect treatment.

40. The method according to claim 39, further comprising the step of retrieving the miniature device at a specified retrieval site.

41. The method according to claim 39, where the entry location is the cisterna magna.
